(19) United States
(12) Patent Application Publication
(10) Pub. No.: US 2015/0354806 A1
(43) Pub. Date: Dec. 10, 2015

(54) HOSPITAL SUPPLY DEVICE WITH PATIENT READING LIGHT

(71) Applicants: ZUMTOBEL LIGHTING GMBH, Dornbirn (AT); ZUMTOBEL LIGHTING GMBH, Lemgo (DE)

(72) Inventors: Bogna LUDWICZAK, Opfenbach (DE); Lukas OSL, Bregenz (AT); Harald SCHWARZER, Maeder (AT); Armin GROM, Waldems-Bermbach (DE)

(21) Appl. No.: 14/759,296

(22) PCT Filed: Jan. 8, 2014
(86) PCT No.: PCT/EP2014/050230

§ 371 Date: Jul. 6, 2015

(30) Foreign Application Priority Data
Jan. 8, 2013 (DE) 10 2013 200 142.8

(51) Int. Cl. F21V 33/00; A61B 19/00; F21V 21/14; F21S 2/00; F21V 23/04

(52) U.S. Cl. CPC F21V 33/0068; F21S 2/00; F21V 23/04; F21V 21/14; A61B 19/5202; F21Y 2101/02

(57) ABSTRACT

In a hospital supply device with an elongated, substantially horizontally extending supply housing with a connector for an electrical power supply and/or for the provision of technical gases. At least one a patient reading light is arranged on an underside of the supply housing. The patient reading light is formed by a number of light modules arranged next to one another in the longitudinal direction of the supply housing, of which a subgroup corresponding to the desired position of the reading light can be activated in a reading light mode.

The present invention relates to a hospital supply device according to the precharacterizing clause of claim 1, having an elongate supply housing, essentially extending horizontally, with connection means for the electrical power supply and/or the provision of technical gases, wherein at least one unit for providing a patient reading light is furthermore arranged on the supply housing.

Such supply devices are used to make the media most often required for the patient treatment available as standard. Besides electricity, these media are in particular oxygen, vacuum and compressed air, which are used in a multiplicity of treatments or examinations, or are often required by the corresponding apparatuses. Since these media are used very often, it would be too complicated to provide a corresponding storage container each time in the individual case. Instead of this, such permanently installed supply devices are installed as standard in patient rooms in the vicinity of the positions for the hospital beds, these being connected to central storage containers for the media and providing corresponding connections so that the media are available at any time.

One configuration which occurs very often is that the housing of the supply device extends horizontally, specifically on the same wall as that on which the patient beds are arranged. In this case, the supply device is then often used not only to make the aforementioned supply media available, but furthermore has lighting means. These may, on the one hand, be means for room lighting, which then emit light on the upper side, for example in order to illuminate the wall or the ceiling of the room in the sense of indirect lighting. On the other hand, in addition, units for providing a so-called patient reading light may also be arranged on the lower side of the supply housing. These are lighting means which ideally emit light in a very limited region, this region corresponding to the position of the patient bed, so that a person lying thereon may for example read without this causing disturbance to other people in the room. To this end, the position of the bed should be adapted to the reading light, or the reading light must be located at the positions of the supply unit, where the beds are then subsequently placed.

During the planning of a patient room, the number of beds provided in the room and the positions at which they are located are generally established relatively early on. The position of the patient reading light on or in the supply housing of the hospital supply device may be established in accordance therewith. On the other hand, a certain flexibility in the positioning of the patient reading light is thoroughly desirable. For example, it would be conceivable for the position of the beds to be slightly modified in the course of time or—at least temporarily—for the number of patient beds to be increased in order to be able to accommodate more patients. In the case of a patient reading light with a fixed position, however, the problem then arises that the lighting sometimes no longer corresponds to the position of the beds and correspondingly does not satisfy requirements. Although solutions in which the displaceable modules are used for providing a patient reading light on the supply housing are known from the prior art, such solutions are however very elaborate and also suffer from problems in terms of the possibility of cleaning them.

The object of the present invention is therefore to provide a new solution for producing a hospital supply device, in which a patient reading light which offers greater flexibility in terms of its positioning is provided.

The object is achieved by a hospital supply device which has the features of claim 1. The dependent claims relate to advantageous refinements of the invention.

The solution according to the invention is based on the concept of forming a unit for providing a patient reading light by using a plurality of modules, which are arranged next to one another in the longitudinal direction of the supply housing. These modules can respectively be activated individually and they extend over a sizeable region, which is in particular greater than the length of a typical patient reading light. That is to say a larger region than is required could be lit by the modules together, although only a subgroup, corresponding the desired position of the reading light, of the light modules can then be activated for the reading light. Because the position of the group within the larger group of light modules can be selected, the position of the patient reading light can be varied at least within a certain range.

The invention therefore provides hospital supply device, which has an elongate supply housing extending horizontally, with connection means for the electrical power supply and/or the provision of technical gases, wherein at least one unit for providing a patient reading light is arranged on a lower side of the supply housing, and according to the invention the unit for providing the patient reading light is formed by a plurality of light modules arranged next to one another in the longitudinal direction of the supply housing, of which a subgroup corresponding to the desired position of the reading light can be activated in a reading light mode.

The solution according to the invention thus makes it possible to vary the position of the patient reading light at least in a certain range. This advantage, however, is achieved without the need for a displaceable module or the like, and is correspondingly simpler to produce in mechanical terms. Because no mobile elements are provided on the supply housing, another advantage is that the supply device can be configured in a substantially closed fashion, which offers significant advantages in terms of cleaning work or the like.

The subgroup activatable as a reading light preferably consists of at least two, in particular four, light modules arranged next to one another, the unit for providing the patient reading light consisting of in total five or seven light modules. Depending on how large the number of modules and their individual size are, the degree of freedom for displacement of the reading light can be varied. On the other hand, too many units which can be driven separately from one another should not be used, since this would in turn entail greater outlay in terms of electronics. The numbers indicated for the subgroup, as well as the light modules used overall to produce the unit for providing the patient reading light represent the optimal compromise in this regard.

Preferably, the activation of the reading light and the adjustment of the position of the reading light are not carried out using the same operating element, i.e. a separate operating element is available for the patient, by means of which he or she can merely switch the reading light on and off, and optionally dim it The adjustment of the position of the reading light, conversely, is preferably carried out by means of a second operating element separate from the first operating element, which is primarily intended for operation by the care staff or the like. Use of the reading light by the end user, i.e. the patient, is facilitated by means of this.

Furthermore, according to the invention, all the light modules of the reading light may be activatable in a further operating mode. In this case, lighting in the region of the patient bed is then obtained which extends over a sizeable distance, which may be very expedient or desirable in certain situations. It would, for example, be suitable to activate all the light modules during a treatment or an examination by the doctor, in order to obtain optimal lighting of the entire patient bed region in this case. The activation of this examination mode is then in turn preferably carried out by means of an operating element provided separately from the operating element for the patient.

According to another particularly preferred embodiment of the invention, the light modules for the patient reading light are formed by LED modules. In comparison with previously known solutions in which fluorescent lamps or the like have been used, light modules based on LEDs have significant advantages. For instance, a lifetime of LEDs is significantly greater, which leads to a reduction of repair or maintenance work. On the other hand extremely compact, narrow, light modules can be formed with the aid of LEDs. In particular, this also opens up the possibility of also arranging the connection means for the electricity supply, i.e. plug sockets and the like, in the same plane as the means for providing the patient reading light and in parallel therewith on the housing of the supply unit.

According to an advantageous refinement of the invention, a so-called accent light may additionally be generated on the lower side of the supply housing. To this end, corresponding means may be arranged in parallel with the LED light modules, although the means for generating the accent light preferably extend essentially over the entire length of the supply housing. Furthermore, a plurality of lighting means, with the aid of which lighting can be achieved in the manner described in the introduction, may be provided on the upper side of the supply housing. These room lighting means are thus configured in order to illuminate the wall or ceiling of the patient room.

Of course, a plurality of units for providing the patient reading light may be provided in the hospital supply device according to the invention. These are then preferably arranged separately from one another in the longitudinal direction of the supply unit. In this case, in contrast to the accent light means, provision is not made for light modules for the patient reading light to extend over the entire length of the supply unit. Such a variant, in which the light modules cover the entirety of the arrangement, would however be conceivable. In this case, provision would then be made that not only the position(s) of the subgroup(s) which form the reading light, but also the number of activatable subgroups, can be selected. There would then be the possibility of adapting the number of reading lights flexibly to the number of beds.

Lastly, it should be pointed out that the use of LED modules for producing a patient reading light may also be employed independently of the inventive concept of the flexible positioning of the reading light, as described above. A further independent claim correspondingly relates to this concept of using LED modules.

The invention will be explained in more detail below with the aid of the appended drawings, in which:

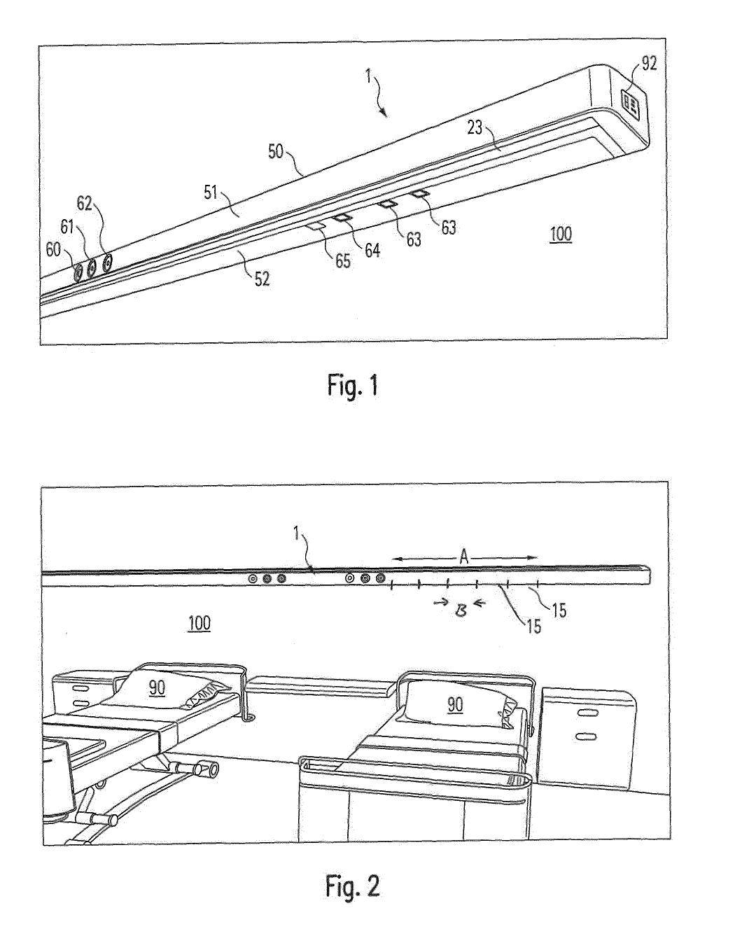

FIG. 1 shows a partial view of a hospital supply device according to the invention in a perspective view;

FIG. 2 shows the arrangement of the hospital supply device in a patient room;

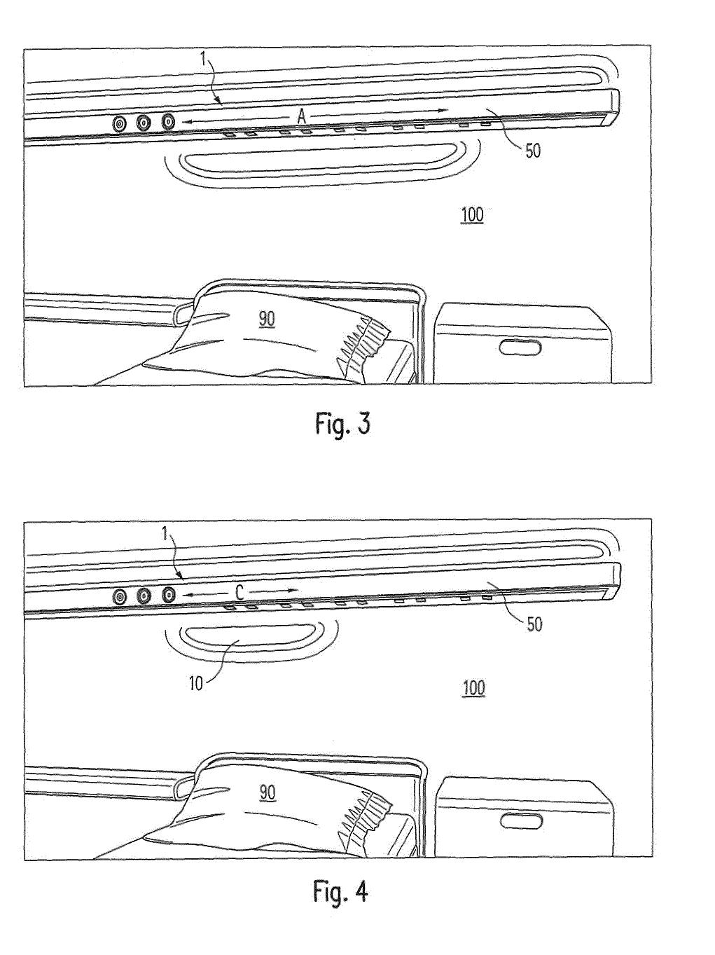

FIG. 3 shows the hospital supply device in a state in which all the light modules of the patient reading light are activated;

FIG. 4 shows the hospital supply device according to the invention in a reading light mode;

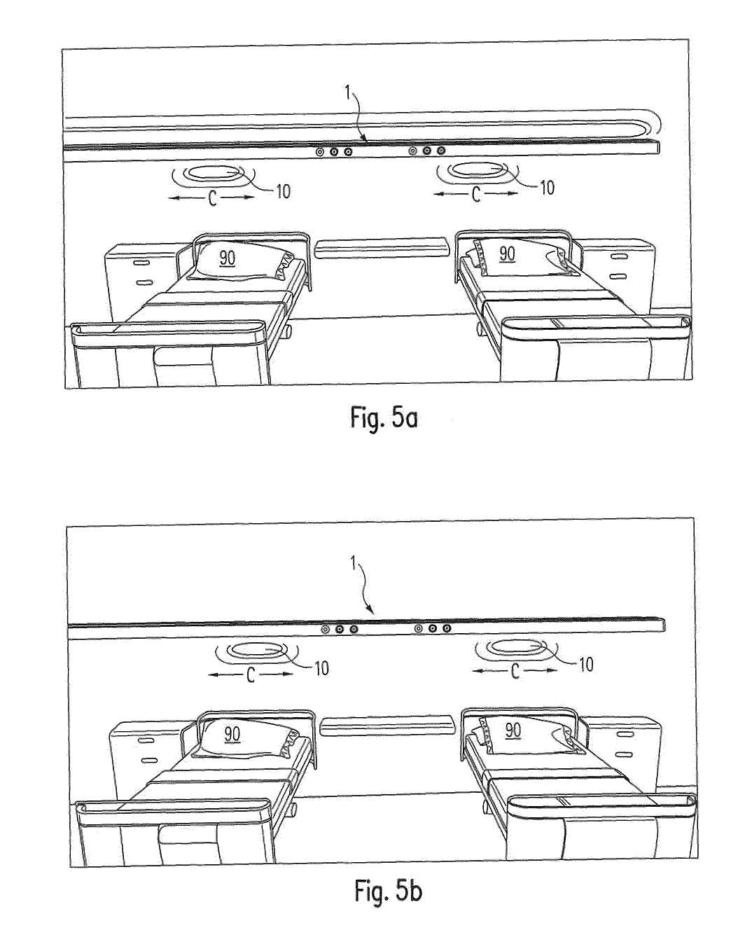

FIGS. 5a to 5c show representations to illustrate the flexibility of the positioning of the patient reading light;

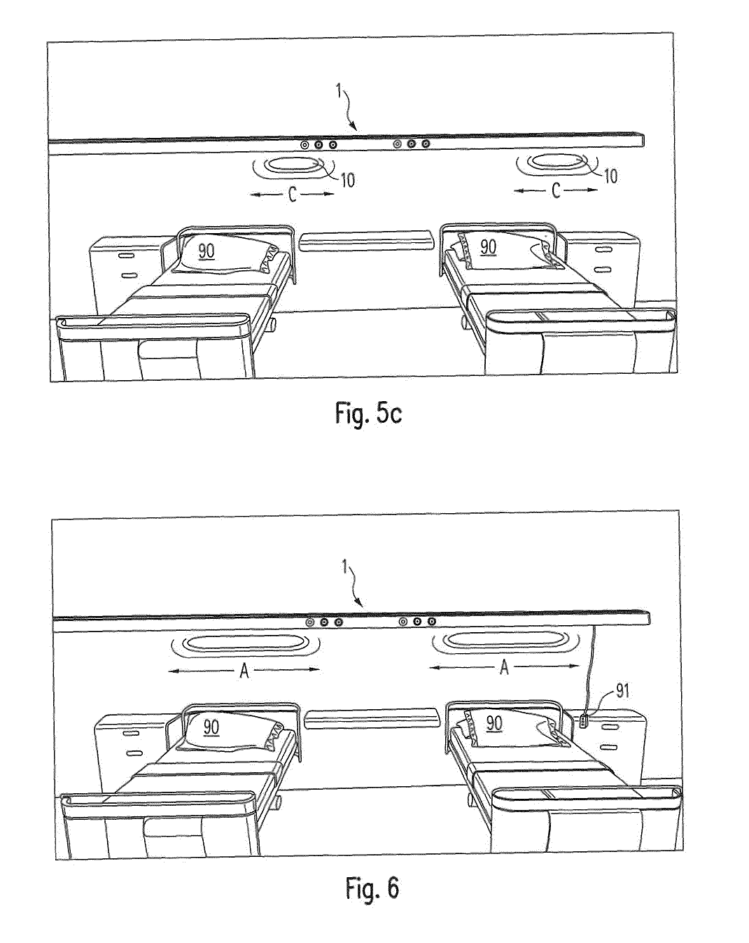

FIG. 6 shows the patient reading light in an examination/treatment mode;

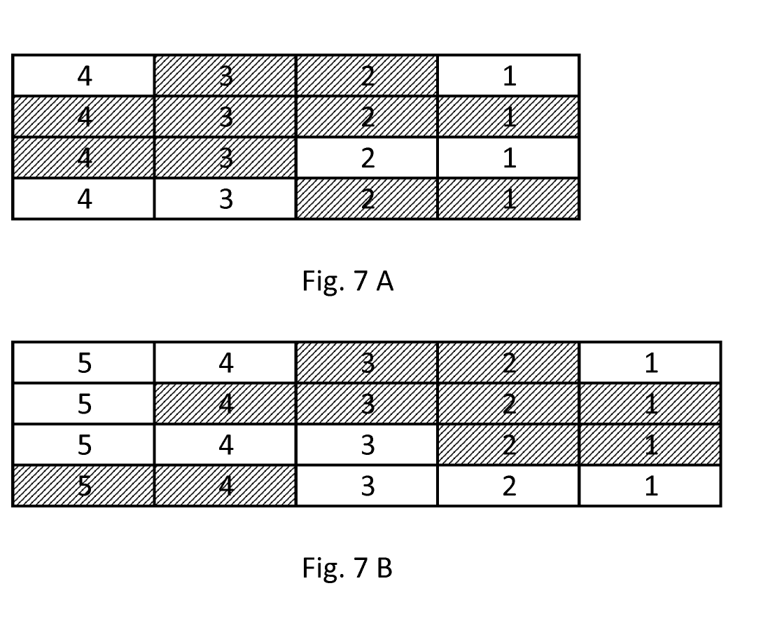

FIGS. 7a and 7b show a schematic representation of the possibility of adjusting the patient reading light;

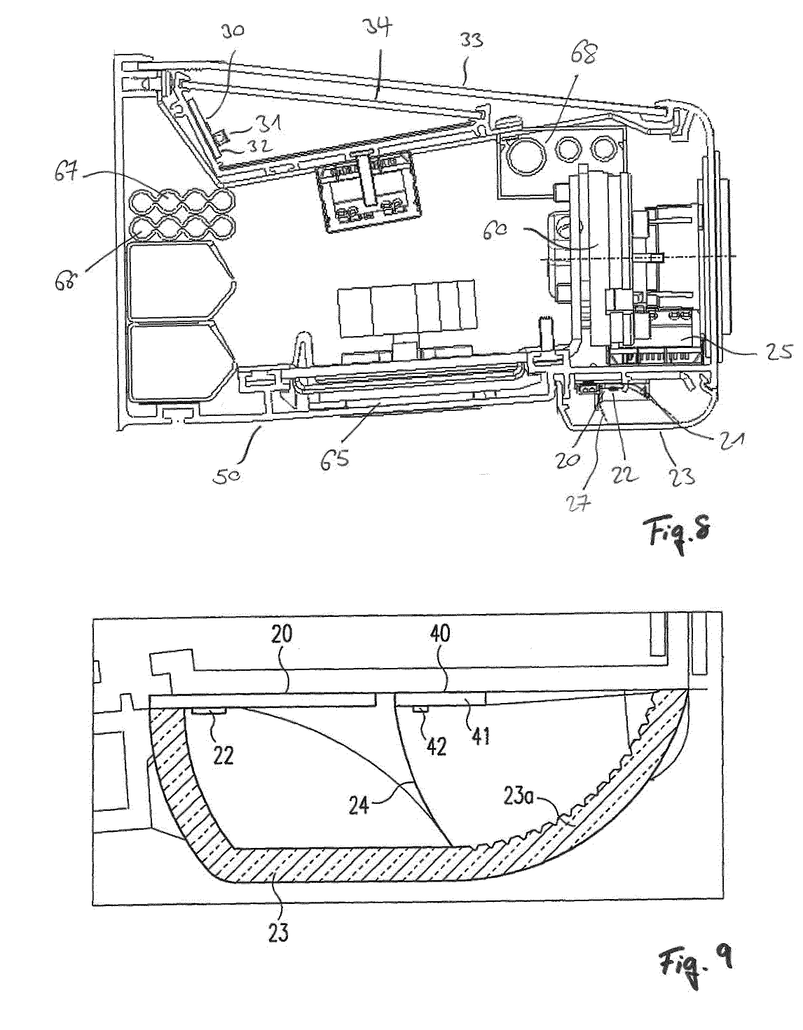

FIG. 8 shows a conceivable embodiment of the hospital supply device according to the invention in a sectional representation and

FIG. 9 shows a variant for the production of the reading light with an accent light located next to it.

The hospital supply device represented in FIG. 1 and provided overall with the reference 1 consists of an elongate supply unit 50 which is arranged, as represented, horizontally on the wall 100 of a patient room. The supply housing forms an elongate channel, in which various lines for providing supply media and electricity extend. Data lines for telecommunication or other applications may also extend inside the housing 50. Connections 60, 61 and 62 for providing technical gases are arranged on the front side 51 of the housing 50. These connections 60 to 62 may then be connected to apparatuses which, for example, require oxygen, vacuum or compressed air for a patient treatment.

Further connections for the electricity supply or for communication 63, 64 and 65 are arranged on the lower side 52 of the supply housing 50, as has usually been the case to date with such devices. Since now, however, as described in more detail below, the means for providing the patient reading light and the means optionally provided for the accent light are based on LEDs, they can be made very narrow compared with earlier solutions, the effect of which is that sufficient free space or room for arranging the connections 63 to 65 is available as before, as represented, on the lower side 52. While it has previously only been possible to position the means for light output and such connections in alternation, now because of the narrow design of the light output means both components can be arranged in parallel, or next to one another. That is to say, the light output means may extend over the entire length of the housing 50, and the connections 63 to 65 can nevertheless be positioned freely on the lower side 52 of the supply housing 50. These connections are then used, as mentioned above, for the electricity supply of electrical apparatuses and for the connection e.g. of a telephone and/or a patient operating element.

The configuration of the connections 60 to 65 and the arrangement of the corresponding lines in the interior of the supply housing 50 are not of further relevance to the present invention, and will therefore not be explained in further detail below. Instead, now in particular the configuration of the patient reading light made available by the supply device 1 will be explained. This light is output through a narrow light exit opening 23 on the lower side of the supply housing 50; this light exit opening 23 may at the same time also be used for the light output of a so-called accent light. In FIG. 1, the light exit opening 23 is represented as illuminated over the entire length of the housing 50, which is attributable to the effect of the accent light, with the aid of which an elongate, weakly illuminating strip is intended to be achieved. The reading light itself, on the other hand, is merely intended to be output over subsections of the housing 50, and according to the invention the position of the reading light can be varied.

The basic concept of the flexible reading light will be explained below with the aid of FIG. 2. In this case, the arrangement of the hospital supply device 1 is firstly represented with respect to two patient beds 90, which are arranged on the same wall 100 as that on which the supply device 1 is arranged. With the aid of a reading light, light output toward the lower side is respectively intended to take place in a relatively narrow region above the corresponding bed 90, in such a way that the patient in the corresponding bed can for example read. Since, however, the position of the bed 90 could also be varied, provision is made for the patient reading light to be formed by a plurality of—schematically represented—light modules 15 arranged next to one another, which in total extend over the length A. In the example represented, it is assumed that in total five modules 15 are used, which are respectively configured identically, i.e. they have the same length B.

Full activation of all the modules 15 would thus achieve light output over the relatively wide range A, which is not absolutely necessary for a reading light per se. According to the invention, therefore, in a reading light mode not all the light modules 15, but only a subgroup thereof, are activated, each subgroup and therefore the position of the reading light being selectable.

The difference in lighting resulting therefrom can be seen by comparing FIGS. 3 and 4. FIG. 3 shows full activation of all the light modules 15, i.e. over the entire region A, while in contrast only a subgroup consisting of two modules 15 is switched on in FIG. 4. In FIG. 4, a reading light 10 is thus generated over the region C, and leads to lighting more in the lateral region of the patient bed 90. It can also be seen in FIGS. 3 and 4 that continuous light is additionally output on the upper side of the supply unit 1, in order in particular to illuminate the wall 100 located above. This indirect lighting provides room lighting, which ensures suitable brightness inside the room but is not suitable for reading.

The flexibility obtained by the concept according to the invention in the production of the patient light is made clear with the aid of FIGS. 5a to 5c. In this case, different positions of the reading light 10 are provided by the selection of corresponding subgroups; in the case represented, the reading light 10 “moves” from left to right in the sequence of the figures, the position of the bed 90 having been kept unchanged for better illustration of the effect. In the present example, two mutually separate units are thus provided for providing a patient reading light 10, each of which individually makes a reading light available respectively for a location of a bed 90. Of course, the position of the reading light 10 can in this case be adjusted individually for each patient bed 90.

The operation and adjustment of the reading light 10 according to the invention are preferably carried out using at least two mutually separate operating elements. A first—only represented in FIG. 6—operating element 91 is in this case made available to the patient at the bed 90 and is used only for switching the reading light on and off, and optionally for dimming it. The operating element 91 is located in the immediate vicinity of the patient bed 90 and is therefore easy for the patient to reach, or operate, but it does not allow any adjustment of the reading light 10. This position adjustment is preferably carried out using a separate operating element 92, which could for example be arranged at an extreme end of the supply unit 1—see FIG. 1—and the operation of which is customarily carried out by the care staff or the like. Patients are therefore not overburdened with numerous different functionalities in the operation of the reading light but can use it in the normal way. Nevertheless, the position of the reading light may be modified by the treatment staff according to the positioning of the beds 90.

Furthermore, it would also be conceivable to activate all the light modules 15 in certain situations, as represented in FIG. 6. In this case, there is lighting over a large range, which may be expedient when an examination or treatment is to be carried out on the patient. The activation of this examination/treatment mode for the lighting may be carried out by means of the operating element 92 or a further operating element, this setting again preferably being available not to the patient but only to the care staff, or the doctor.

FIGS. 7a and 7b further illustrate the adjustment possibilities which may for example be carried out by means of the operating element 92 in respect of the positioning of the reading light. FIG. 7a represents four modules arranged next to one another, one of the four configurations represented being selectable by corresponding switch setting or actuation of a selection element of the operating element 92. These are arranged under one another, so that in the first switch setting the two central modules form the reading light. In the second switch setting, conversely, all four modules are activated, which may for example correspond to the examination/treatment mode. Selection options three and four in turn allow positioning of the patient light at the left or right end of the light module group.

FIG. 7b shows a corresponding representation for the case in which a light module group consists of in total 5 units. Again, four different configurations corresponding to four switch representations are shown, a reading light which is positioned offset to the middle right being formed in the first setting. The second switch setting corresponds to the examination/treatment mode, in which the lighting is activated over four units of the light module group. The further settings in turn result in a reading light at the right or left end of the arrangement.

As already mentioned, provision is preferably made that such a setting cannot be carried out by the patient himself or herself but, for example, by the examining doctor or the care staff The patient himself or herself is then capable of switching the selectable and therefore activatable units on or off, and optionally dimming them.

In the exemplary embodiment represented, the units for providing the patient reading light are arranged separately from one another, i.e. there is a distance between them. It would, however, also be conceivable to arrange these units directly next to one another, so that the light modules for the patient reading light optionally extend over the entire length of the supply unit. In this case, provision may then in addition be made that not only the position(s) of the subgroup(s) which form the reading light, but also the number of activatable subgroups, can be selected. Such input, which is again preferably available only to staff, i.e. must be carried out by means of a correspondingly separate operating element, would then offer the possibility of adapting the number of reading lights flexibly to the number of beds.

One conceivable embodiment of a supply device according to the invention is shown in sectional representation in FIG. 8, the housing 50 being formed by an elongate profiled body which is used to accommodate the various units. For example, the connection units 60 and 65 arranged inside the profiled body for providing technical gases and/or electricity can be seen. Lines 66 and 67, extending inside the longitudinal channel formed by the profiled body, for the electricity and data as well as for the technical gases are furthermore represented. As mentioned above, the arrangement of these components inside the housing 50 has a subordinate role for the present invention and is therefore not described in detail.

In the exemplary embodiment of FIG. 8, an accent light is not provided; rather, only a reading light and a light intended for room lighting are provided. The reading light is in this case formed by the aforementioned light modules arranged next to one another, which are preferably each formed by an LED module 20 that comprises a circuit board 21 with LEDs 22 arranged thereon. The light output in this case takes place toward the lower side via a light exit lens 23 configured to be slightly curved, which extends over the entire length of the housing 50 and, for example, is provided with optical structures such that the LEDs 22 are not perceptible as individual light points. With the aid of such optical structures, it is also possible to avoid transition regions between switched-on light modules being perceptible when activating a corresponding group of light modules or when activating the examination/treatment mode. In the exemplary embodiment represented, as schematically represented, additional optical elements 27 are furthermore provided in the space enclosed by the light exit lens 23, for example grating reflectors or prism elements, with the aid of which the direction of the light output can be influenced in a desired way. By means of corresponding operating apparatuses 25, the individual LED modules 20 can then be driven in the manner described above.

The indirect lighting or the room light is likewise carried out by means of LED modules 30, which are arranged in an upper free space of the housing 50. The LEDs 31 are in this case arranged on a circuit board 32, or plurality of circuit boards 32, arranged at an inclination, the light output by the LEDs 31 being emitted through a diffuse light exit lens 33. The LED lighting inserts for the indirect lighting are additionally protected by a transparent lens 34, which covers the reception region for the circuit boards 32 and thus forms protection against contact.

If an accent or effect light is also intended to be made available in addition to the reading light, an arrangement of light sources as represented in FIG. 9 may be provided. Again, the aforementioned LED modules 20 with the LEDs 22 are provided for generating the reading light. Next to these, however, further light sources 40 are now provided, which are in turn formed by circuit boards 41 with LEDs 43 arranged thereon, by means of which the accent light is to be generated. Both light sources 20, 40 are arranged inside a jointly used optical element 23, but they are separated from one another by a preferably reflective partition wall 24. That is to say, although the different light sources 20, 40 use the same light emission element 23, they use different regions thereof; furthermore—as can be seen—the light emission region for the accent light may be provided with additional structuring 23a, which may be advantageous with a view to the desired light output, namely achieving a weakly lit strip over the entire length of the supply unit 1. In this variant as well, however, a very compact, in particular narrow design is achieved, so that as before—as also represented in FIG. 8—connection means for the electricity supply and data transmission can be provided laterally next to the light sources 20, 40 for the reading light and optionally for the accent light on the lower side of the housing 50.

Ultimately, by the measures according to the invention, the properties of a hospital supply device are substantially improved, particularly in respect of the light output thereby achievable. The solution according to the invention makes it possible, in particular, to adapt the position of a reading light flexibly so that there is much greater freedom in the positioning of patient beds.

1. A hospital supply device having an elongate supply housing, essentially extending horizontally, with connection means for the electrical power supply and/or the provision of technical gases, wherein at least one unit for providing a patient reading light is arranged on a lower side of the supply housing, wherein the unit for providing the patient reading light is formed by a plurality of light modules arranged next to one another in the longitudinal direction of the supply housing, of which a subgroup corresponding to the desired position of the reading light can be activated in a reading light mode.

2. The hospital supply device as claimed in claim 1, wherein the subgroup activatable as a reading light comprises at least two, preferably four, light modules arranged next to one another, the unit for providing the patient reading light consisting of in total five or seven light modules.

3. The hospital supply device as claimed in claim 1, wherein it has at least a first operating element for switching the reading light on or off, and optionally for dimming, as well as a second operating element, separate from the first operating element, for adjusting the position of the reading light.

4. The hospital supply device as claimed in claim 1, wherein in a further operating mode, all the light modules of the unit providing the patient reading light can be activated.

5. The hospital supply device as claimed in claim 1, wherein the light modules are formed by LED modules.

6. The hospital supply device having an elongate supply housing, essentially extending horizontally, with connection means for the electrical power supply and/or the provision of technical gases, wherein at least one unit for providing a patient reading light is arranged on a lower side of the supply housing, wherein the unit for providing the patient reading light is formed by a plurality of light modules arranged next to one another in the longitudinal direction of the supply housing, which are respectively formed by LED modules.

7. The hospital supply device as claimed in claim 5, wherein additional means for generating an accent light are arranged on the lower side of the supply housing.

8. The hospital supply device as claimed in claim 7, wherein the means for generating an accent light extend essentially over the entire length of the supply housing, and in particular are arranged parallel to the unit for providing the patient reading light.

9. The hospital supply device as claimed in claim 7, wherein it has a plurality of units providing a patient reading light, which are preferably arranged separately from one another in the longitudinal direction of the supply housing.

10. The hospital supply device as claimed in claim 7, wherein additional lighting means for room lighting are arranged on the upper side of the supply housing, which means give rise in particular to upwardly directed light output and are preferably arranged continuously over the entire length of the supply housing.
